(19) United States
(12) Patent Application Publication
(10) Pub. No.: US 2026/0108279 A1
(43) Pub. Date: Apr. 23, 2026

(54) SPINAL ALIGNMENT SYSTEM, RECEIVER, AND METHOD THEREOF

(71) Applicant: Thomas J. Errico, Coral Gables, FL (US)

(72) Inventor: Thomas J. Errico, Coral Gables, FL (US)

(21) Appl. No.: 19/367,686

(22) Filed: Oct. 23, 2025

Related U.S. Application Data
(60) Provisional application No. 63/710,700, filed on Oct. 23, 2024.
(60) Provisional application No. 63/768,438, filed on Mar. 7, 2025.

(51) Int. Cl. A61B 17/70; A61B 17/86

(52) U.S. Cl. CPC A61B 17/7074; A61B 17/7008; A61B 17/7032; A61B 17/7038; A61B 17/8605

(57) ABSTRACT

Disclosed is a spinal alignment system that includes a receiver having first and second pair of legs coupled to each other by way of a plurality of hinged connections that enables the first and second pair of legs to exhibit one of, a neutral position or a contracted position. One or more tabs disposed onto an inner surface of each leg of the first and second pair of legs, a retaining means that is adapted to couple at least one leg of the first pair of legs with at least one leg of the second pair of legs to retain the first and second pair of legs in the neutral position, and a pedicle screw such that a head portion of the pedicle screw is inserted and held in a first opening of the receiver. A rod adapted to be inserted inside the second opening.

### CROSS REFERENCE

This patent application claims the benefit of priority to U.S. Provisional Application 63/768,438 filed 7 Mar. 2025 and U.S. Provisional Application 63/710,700 filed 23 Oct. 2024; each of which is incorporated by reference herein in its entirety for all purposes.

### TECHNICAL FIELD

The present disclosure relates to a medical equipment, more particularly the present disclosure relates to a spinal alignment system, a receiver, and a method thereof.

### BACKGROUND

Spinal immobilization has been known and used for many years in the treatment of spinal instability and displacement. Various methods of internal and external fixation have been developed over time with the increase in sophistication of surgical techniques. In external fixation, at least some portion of the stabilization device is involved which is external to the patient's body. On the other hand, in internal fixation, methods of stabilization which are wholly internal to the patient and devices such as screws, plates and rods are involved.

In some of the methods of internal fixation, bone screws are utilized in spinal surgeries in order to secure various implants to vertebrae along the spinal column for the purpose of stabilizing and/or adjusting spinal alignment. Specifically, pedicle screws are commonly used in surgeries today for spinal alignment. The existing pedicel screw contains a head and a shaft coupled to the head. The existing pedicle screw further engage a receiver for receiving a separate rod that is further coupled to other receivers engages by other pedicle screws that are placed in adjacent vertebrae for providing stabilization. However, the existing receivers are not able to perform spinal fixation efficiently.

Therefore, there is a need for a technical solution that overcomes the aforementioned problems of the state of art receivers for pedicle screws of the conventional spinal alignment systems.

### SUMMARY

In an aspect of the present disclosure, a receiver for a spinal alignment system is disclosed. The receiver includes a first outside portion and a second inside portion. The first outside portion includes a first top and a first bottom, and the second inside portion includes a second top and a second bottom. The first outside portion includes a first pair of legs and a first upper portion and the second inside portion includes a second pair of legs and a second upper portion. A first leg of the first pair of legs is coupled to a second leg of the second pair of legs, and a third leg of the first pair of legs is coupled to a fourth leg of the second pair of legs. In certain embodiments, each of the legs has a distal end and a proximal end. In certain embodiments, each of the legs may be coupled to each other by way of a hinge connection. In certain embodiments, the hinge connection is positioned at or at about the distal end of each leg, at or at about the proximal end of each leg, at or at about the middle of each leg or anywhere in between. Each of the first leg is coupled to the second leg and the third leg is coupled to the fourth leg by way of a hinged connection that enables the first pair and the second pair of legs to each exhibit one of, a first position or a second position relative to each other.

The receiver includes at least a first tab disposed onto an inner surface between the first pair of legs and at least a second tab disposed onto an inner surface between the second pair of legs, aligned with and opposite the at least the first tab.

In certain embodiments, each of the tabs may be positioned at about or at the top of the legs, at about or at the bottom of the legs, at about or at the middle of the legs or anywhere in between.

In certain embodiments, each of the tabs may be positioned above the legs, as shown in FIG. 1D, or below the legs, as shown in FIG. 1E. In these configurations, there can be one or more extensions that extend above the legs or below the legs to support the tabs.

Furthermore, the receiver includes at least one retainer positioned at or at about the proximal end and coupled to the first upper portion and the second upper portion. The retainer may be in the form of a pin, a joint, a gear, a screw, hinge, or some other connective element. In certain embodiments, the receiver includes a plurality of retainers, with at least one retainer positioned at the first upper portion and the second upper portion. Each retainer is adapted to couple the first upper portion with the second upper portion to retain the first upper portion and the second upper portion in at least one of the first position or the second position.

In certain embodiments, when the first pair of legs and the second pair of legs are in the first position a first opening is defined between the first tab and the second tab that results in the receiver forming a closed or locked position relative to a crown of a pedicle screw. The term “crown” is used herein interchangeably with the term “head” and is referring to the top portion of the pedicle screw, via which a tool engages with the pedicle screw to screw the pedicle screw into a bone or other anatomical element.

In some embodiments of the present disclosure, when the first pair of legs and the second pair of legs are in the second position relative to each other, a first opening is defined between the first tab and the second tab that results in the receiver forming an open or unlocked position relative to the crown of the pedicle screw.

In certain embodiments, the unlocked position can be achieved by compression of the first upper portion of the first outside portion and the second upper portion of the second inside portion towards each other, i.e., when they are moved to a contracted position. In other embodiments, the unlocked position can be achieved by expansion of the first upper portion of the first outside portion and the second upper portion of the second inside portion away from each other, i.e., when they are moved to an expanded position.

In certain embodiments of the present disclosure a second opening and a third opening are formed between the first outside portion and the second inside portion, above the first tab and second tab and below the first top and the second top. A first lower portion of the second opening and a second lower portion of the third opening are located above and adjacent to the top of the first tab and second tab. A first upper portion and a second upper portion of the second opening and the third opening are located below the first top and the second top.

In certain embodiments, the receiver includes a fourth opening positioned between the first top and the second top.

In some embodiments, a spring or flexion member is placed between the first outside portion and the second inside portion such that upon movement of the first upper portion and the second upper portion toward or away from each other, the spring is compressed, flexed or tensioned.

In some embodiments of the present disclosure, the first opening is adapted to receive the head portion of the pedicle screw when a force is placed on the first upper portion and the second upper portion to move them toward each other into a contracted position or away from each other into an expanded position. Upon release or removal of the force upon the first upper portion and the second upper portion, the first and second pair of legs automatically return to the first position via the spring or flexion member, and the receiver, via the first tab and the second tab, holds the head portion within the first opening.

In some embodiments of the present disclosure, the second opening and the third opening are is adapted to receive a rod such that the rod is engaged by way of the rod entering into the receiver through the top of the receiver via the fourth opening when the first and second pair of legs are in the neutral position.

In some embodiments of the present disclosure, a third tab may be disposed at or at about the top of the first upper portion and a fourth tab may by disposed at or at about the top of the second upper portion on the inner surface of each of the first upper portion and the second upper portion.

In some embodiments of the present disclosure, the fourth opening is adapted to receive a cap having an open center portion such that the cap is attached to an outer face of the first upper portion of the first outside portion and an outer face of the second upper portion of the second inside portion. The attachment of the cap may be accomplished via grooves or holes in the outer face of the first upper portion and the second upper portion and corresponding extensions on the inside of the cap. Alternatively, the attachment of the cap may be accomplished via threads on the outer face of the first upper portion and the second upper portion and corresponding threads on the inside of the cap. In certain embodiments, the cap may have an inside border that extends over the inside edge of the top of the outside portion and the top of the inside portion to prevent them from moving inwardly and causing an unexpected release of the cap.

In certain embodiments, in place of a cap, a ring is positioned around and within grooves on the outer face of the first upper portion and the second upper portion. Alternatively, there may be holes around the outer face of the first upper portion and the second upper portion and the ring includes extensions that correspond to and are positioned within the holes. In other embodiments, the outer face of the first upper portion and the second upper portion include threads that correspond to threads on the inside of the ring and the ring is threaded onto the outer face of the first upper portion and the second upper portion.

In some embodiments of the present disclosure, the first tab and the second tab are in the form of arced upwardly facing lips that are adapted to be engaged with the head portion of the pedicle screw when the head portion is a mushroom shaped head.

In some embodiments of the present disclosure, the device further includes a locking washer through which a threaded portion of the pedicle screw may be inserted such that the locking washer rests proximate to the head portion of the pedicle screw when the receiver is attached to the pedicle screw.

In some embodiments of the present disclosure, the device further includes a c-ring through which the threaded portion of the pedicle screw may be inserted such that the c-ring rests proximate to the head portion of the pedicle screw when the receiver is attached to the pedicle screw.

In some embodiments of the present disclosure, each leg of the first pair of legs of the outside portion and each leg of the second pair of legs of the inside portion has a horizontal portion and a vertical portion such that (i) the horizontal portion of each leg of the first pair of legs of the outside portion and each leg of the second pair of legs of the inside portion comprising at least one through hole (ii) the horizontal portion of each leg of the first pair of legs comprising a slot on an outer or inner surface, (iii) the horizontal portion of each leg of the second pair of legs comprising a slot on an inner or outer surface.

In some embodiments of the present disclosure, the plurality of hinged connections comprising a plurality of wire springs and a plurality of pivot pins, respectively, wherein, to couple the first pair of legs with the second pair of legs, the at least one through hole of the first pair of legs is aligned with the at least one through hole of the second pair of legs such that the at least one through hole receives a wire spring of the plurality of wire springs and a pivot pin of the plurality of pivot pins.

In some embodiments of the present disclosure, each wire spring of the plurality of wire springs comprising a plurality of upright portions and a central portion such that the plurality of upright portions are engaged within the slots provided on the outer or inner surface of the first and second pair of legs when the first and second pair of legs are aligned.

In some embodiments of the present disclosure, a plurality of legs of the first pair of legs are integrally connected to form a U-shaped structure and a plurality of legs of the second pair of legs are integrally connected to form a U-shaped structure.

In some embodiments of the present disclosure, the vertical portion of the plurality of legs of the first pair of legs and the plurality of legs of the second pair of legs comprising a horizontal slot at about the upper end on the outer surface to accept one or more tools therein.

In another aspect of the present disclosure, a spinal alignment system is disclosed. The spinal alignment system includes a receiver, a pedicle screw, and a rod. The receiver includes first and second pair of legs coupled to each other by way of a plurality of hinged connections that enables the first and second pair of legs to exhibit one of, a first position, where the head of he pedicle screw is retained by the receiver, or a second position, where the receiver is open thereby allowing the head of the pedicle screw to move into position within the receiver. The first and second pair of legs in the first position defines first through fourth openings. Further, the receiver includes at least a first tab disposed onto an inner surface of the first outer portion and at least a second tab disposed onto an inner surface of the second inner portion. The receiver may include a retaining means that is adapted to couple at least one leg of the first pair of legs with at least one leg of the second pair of legs to retain the first and second pair of legs in the first position. The pedicle screw has a threaded portion for securing the pedicle screw to a vertebra and a head portion coupled to the threaded portion. The head portion of the pedicle screw is inserted into the first opening when the first and second pair of legs are in the second position such that upon release of the first and second pair of legs, the first and second pair of legs return to the first position and at least the first tab on the first outer portion and at least the second tab on the second inner portion holds the head portion within and prevents the head portion from exiting the first opening. The rod is adapted to be inserted through the fourth opening and extend through the third opening and the fourth opening. The rod may be engaged by way of (i) at least a third tab on the inner surface of the first upper portion and at least a fourth tab on the inner surface of the second upper portion, (ii) a cap, or (iii) a ring, when the first and second pair of legs are in the neutral position.

In some embodiments of the present disclosure, in the second position, the first upper portion and the second upper portion are moved inwards towards each other and (ii) in the first position, the first upper portion and the second upper portion are at the substantially parallel position with respect to one another. In some embodiments, the first upper portion and the second upper portion are prevented from moving outward.

In some embodiments of the present disclosure, in the second position, the first upper portion and the second upper portion are moved outwards away from each other and (ii) in the first position, the first upper portion and second upper portion are at the substantially parallel position with respect to one another.

In some embodiments of the present disclosure, the rod is selected from one of, a cylindrical rod, a rod with one or more flat sides, or a rod having a tapered or trapezoidal cross section.

In some embodiments of the present disclosure, the head portion of the pedicle screw has a flat surface, an arced surface, an upwardly or downwardly curved surface, or a recessed surface.

In some embodiments of the present disclosure, the at least one third tab and the at least one fourth tab are disposed on an inner surface of each of the first upper portion and the second upper portion, respectively.

In some embodiments, the at least one third tab or the fourth tab, or both have a flat bottom surface and a downwardly and inwardly sloping upper surface.

In some embodiments of the present disclosure, the system further includes a cap adapted to be attached to the proximal face of the receiver onto the first upper portion and the second upper portion to enclose the fourth opening.

In some embodiments of the present disclosure, the cap comprising an open center portion.

In some embodiments of the present disclosure, a first tab on or coupled to the outer portion at or at about the first pair of legs and a second tab on or coupled to the inner portion at or at about the second pair of legs in the neutral position each comprises a plurality of upward facing lips at or at about a bottom of the receiver.

In some embodiments of the present disclosure, the head portion of the pedicle screw is a mushroom shaped head comprising a downward facing overhang that engages with an upward facing lips of the receiver.

In some embodiments of the present disclosure, the device further incudes a locking washer through which the threaded portion of the pedicle screw may be inserted such that the locking washer rests proximate to the head portion of the pedicle screw when the receiver is coupled to the pedicle screw.

In some embodiments of the present disclosure, the device further includes a c-ring through which the threaded portion of the pedicle screw may be inserted such that the locking washer rests proximate to the head portion of the pedicle screw when the receiver is coupled to the pedicle screw.

In some embodiments of the present disclosure, each leg of the first and second pair of legs has a horizontal portion and a vertical portion such that (i) the horizontal portion of each leg of the first and second pair of legs comprising at least one through hole (ii) the horizontal portion of each leg of the first pair of legs comprising a slot on an outer surface or an inner surface, (ii) the horizontal portion of each leg of the second pair of legs comprising a slot on an inner surface or an outer surface.

In some embodiments of the present disclosure, the plurality of hinged connections comprise a plurality of wire springs and a plurality of pivot pins, respectively, wherein, to couple the first pair of legs with the second pair of legs, the at least one through hole of the first leg of the first pair of legs is aligned with the at least one through hole of the third leg of the second pair of legs such that the at least one through hole of the first leg of the first pair of legs and the at least one through hole of the third leg of the second pair of legs receives a first wire spring of the plurality of wire springs and a first pivot pin of the plurality of pivot pins, and the at least one through hole of the second leg of the first pair of legs is aligned with the at least one through hole of the fourth leg of the second pair of legs such that the at least one through hole of the second leg of the first pair of legs and the at least one through hole of the fourth leg of the second pair of legs receives a second wire spring of the plurality of wire springs and a second pivot pin of the plurality of pivot pins.

In some embodiments of the present disclosure, each wire spring of the plurality of wire springs comprises a plurality of upright portions and a central portion such that the plurality of upright portions are engaged within the slots provided on the outer surface or inner surface of the first and second pair of legs when the first and second pair of legs are aligned.

In some embodiments of the present disclosure, the first pair of legs are integrally connected to the second pair of legs to form a U-shaped structure.

In some embodiments of the present disclosure, the first leg of the first pair of legs and the third leg of the second pair of legs are integrally connected, and the second leg of the first pair of legs and the fourth leg of the second pair of legs are integrally connected, to form a U-shaped structure from the outer portion and the inner portion.

In some embodiments of the present disclosure, the outside portion and the inside portion comprises a vertical slot beginning at or at about the top of the outside portion and the top of the inside portion.

In some embodiments of the present disclosure, the outside portion and the inside portion comprises a vertical slot at about the top of the outside portion and the top of the inside portion to accept one or more tools therein.

In some embodiments of the present disclosure, the outside portion and the inside portion comprise a fourth opening at or on the top of the outside portion and the top of the inside portion to accept one or more tools therein.

In yet another aspect of the present disclosure, a method for assembling a spinal alignment system is disclosed. The method includes a step of contracting a proximal face of a receiver by applying an external force on first and second pair of legs of the receiver such that the first and second pair of legs are bent towards each other to exhibit a contracted position thus expanding a first opening present on a distal face of the receiver. The contraction is enabled by way of a plurality of hinged connections attaching the first and second pair of legs. Further, the method includes a step of inserting a head portion of a pedicle screw inside the first opening. Furthermore, the method includes a step of releasing the external force applied on the first and second pair of legs such that the first and second pair of legs return to a first position thus holding the head portion of the pedicle screw therein. Furthermore, the method includes a step of inserting a rod inside a fourth opening present in a top central portion of the receiver, wherein the rod extends through a second and third opening between the upper portions of the outside portion and the inside portion of the receiver. Furthermore, in certain embodiments the method includes a step of engaging one or more tabs disposed on an inner surface of each upper portion of the outside portion and the inside portion with the rod. Furthermore, the method may include a step of attaching a cap onto the first upper portion of the outside portion and the second upper portion of the inside portion to enclose the fourth opening present on the proximal face of the receiver.

In some embodiments of the present disclosure, the method further includes a step of engaging the head portion of the pedicle screw having a mushroom shape with a plurality of upward facing lips present at a bottom of the receiver.

In some embodiments of the present disclosure, the method further includes a step of inserting a threaded portion of the pedicle screw through a locking washer such that the locking washer rests proximate to the head portion of the pedicle screw when the receiver is attached to the pedicle screw.

In some embodiments of the present disclosure, the method further includes a step of inserting the threaded portion of the pedicle screw through a c-ring such that the locking washer rests proximate to the head portion of the pedicle screw when the receiver is attached to the pedicle screw.

In some embodiments of the present disclosure, the method further includes a step of engaging a plurality of upright portions of a wire spring within slots provided on the outer surface or inner surface of the first pair of legs and within slots provided on the outer surface or inner surface of the second pair of legs when the first and second pair of legs are aligned.

### BRIEF DESCRIPTION OF THE DRAWINGS

The following detailed description of the preferred embodiments of the present disclosure will be better understood when read in conjunction with the appended drawings. The present disclosure is illustrated by way of example, and not limited by the accompanying figures, in which like references indicate similar elements.

FIG. 1A illustrate a top view, and FIGS. 1B-1C illustrate side views of a spinal alignment system according to an embodiment of the present disclosure;

FIG. 2A illustrates an exploded side view of components of a receiver of the spinal alignment system of FIGS. 1A-1C, according to an embodiment of the present disclosure;

FIG. 2B illustrates an exploded side view of components of a receiver of the spinal alignment system of FIGS. 1A-1C having tabs in a first configuration, according to an embodiment of the present disclosure;

FIG. 2C illustrates an exploded side view of components of a receiver of the spinal alignment system of FIGS. 1A-1C having tabs in a second configuration, according to an embodiment of the present disclosure;

FIGS. 3A-3C illustrate side views of a receiver with a rod, according to different embodiments of the present disclosure;

FIGS. 4A-4B illustrates a side view of a receiver with the pedicle screw, according to an embodiment of the present disclosure;

FIG. 4C illustrates a side view of a receiver with extensions, according to an embodiment of the present disclosure;

FIG. 5A illustrates a bottom view of a receiver, according to an embodiment of the present disclosure;

FIG. 5B illustrates a top view of another receiver, according to an embodiment of the present disclosure;

FIG. 5C illustrates a top view of another receiver, according to an embodiment of the present disclosure;

FIG. 6A illustrates a pedicle screw with a head portion having a flat surface, according to an embodiment of the present disclosure;

FIG. 6B illustrates the pedicle screw with a head portion having a mushroom shape, according to an embodiment of the present disclosure;

FIG. 6C illustrates the pedicle screw with a head portion, according to an embodiment of the present disclosure;

FIG. 6D illustrates the pedicle screw with a head portion, according to an embodiment of the present disclosure;

FIG. 6E illustrates the pedicle screw with a head portion, according to an embodiment of the present disclosure;

FIG. 6F illustrates the pedicle screw with a head portion, according to an embodiment of the present disclosure;

FIG. 6G illustrates the pedicle screw with a head portion, according to an embodiment of the present disclosure;

FIG. 7 illustrates a side view of the receiver having a cap, with a side cross sectional view of the cap, according to an embodiment of the present disclosure; and

FIG. 8 illustrates a flowchart of a method for assembling the spinal alignment system, according to an embodiment of the present disclosure.

FIG. 9A illustrates a top view of a receiver, according to an embodiment of the present disclosure.

FIG. 9B illustrates a side view of a receiver and a pedicle screw, according to an embodiment of the present disclosure.

FIG. 9C illustrates a front view of a receiver and a pedicle screw, according to an embodiment of the present disclosure.

FIG. 9D illustrates a perspective view of a receiver and a pedicle screw, according to an embodiment of the present disclosure.

FIGS. 10A-10C illustrate an assembly of a receiver and a pedicle screw from a side view perspective, according to an embodiment of the present disclosure. In FIG. 10A the receiver is compressed to put it into an insertion mode. In FIG. 10B the pedicle screw is inserted into the receiver, and in FIG. 10C the receiver is released to lock the pedicle screw within the receiver.

FIG. 11A illustrates an enlarged side view of a receiver and a pin, according to an embodiment of the present disclosure.

FIG. 11B illustrates an enlarged top view of a receiver and a pivot pin, according to an embodiment of the present disclosure.

FIG. 12 illustrate an enlarged perspective view of a head of a pedicle screw, according to an embodiment of the present disclosure.

FIG. 13 illustrated a perspective view of an assembled receiver and pedicle screw, according to an embodiment of the present disclosure.

FIG. 14 illustrates an enlarged bottom perspective view of an assembled receiver and pedicle screw, according to an embodiment of the present disclosure.

### DETAILED DESCRIPTION

The detailed description of the appended drawings is intended as a description of the currently preferred embodiments of the present disclosure, and is not intended to represent the only form in which the present disclosure may be practiced. It is to be understood that the same or equivalent functions may be accomplished by different embodiments that are intended to be encompassed within the spirit and scope of the present disclosure.

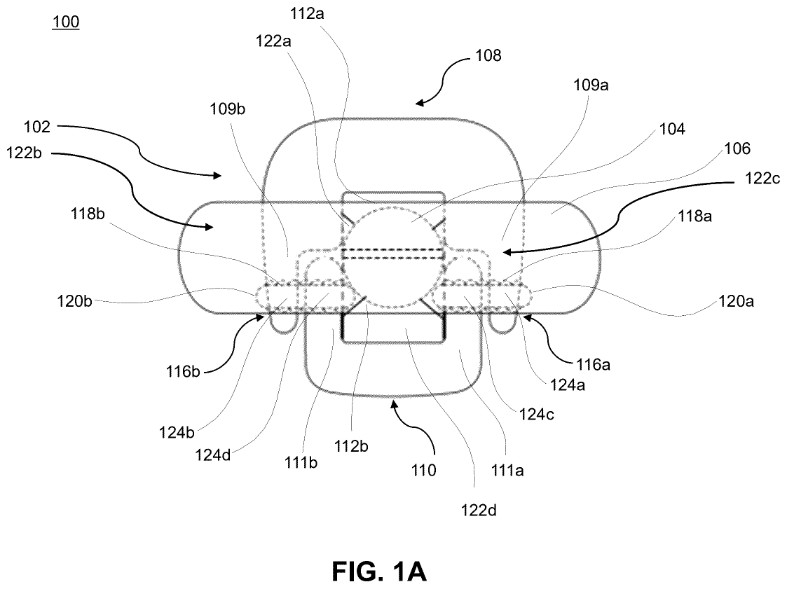

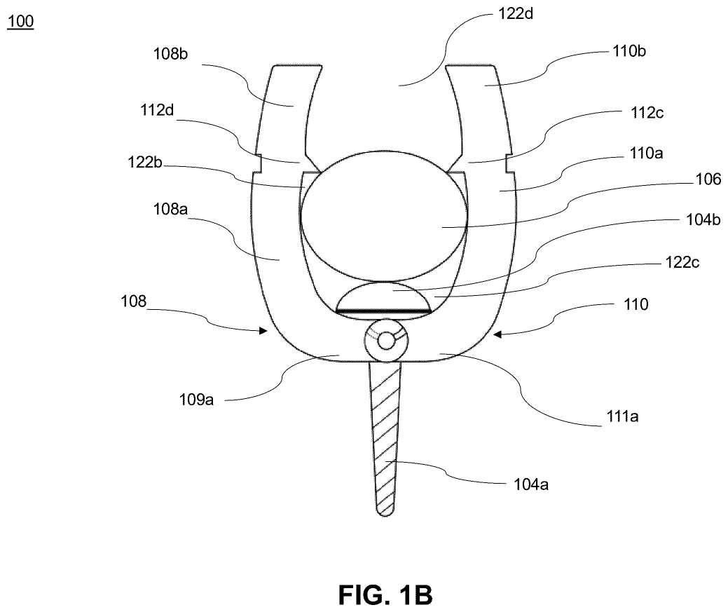

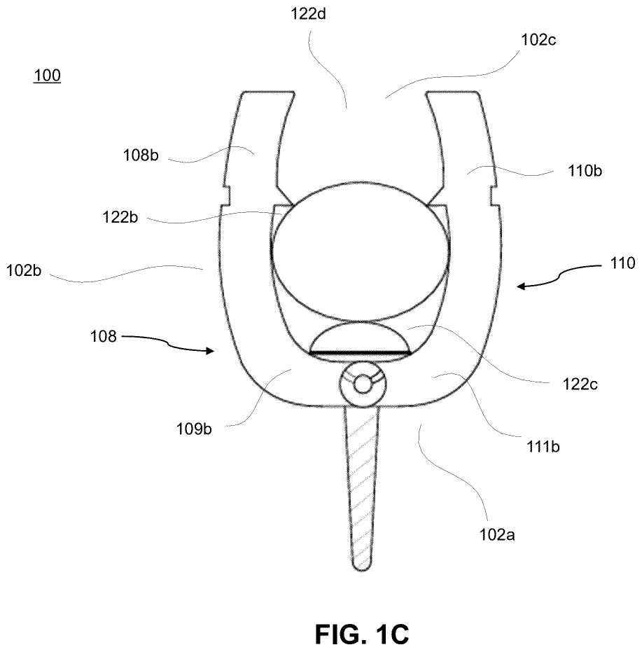

FIG. 1A illustrates a top view of a spinal alignment system 100 and FIGS. 1B-1C illustrate a side views of the spinal alignment system 100, according to an embodiment of the present disclosure. The spinal alignment system 100 may be adapted to facilitate spine surgery by providing high rigidity and resistance to pullout. Specifically, the spinal alignment system 100 may be adapted to engage all three columns of the spine and facilitate to maintain spine alignment, reduction after fractures, and correction of deformities. The spinal alignment system 100 may include a receiver 102, a pedicle screw 104, and a rod 106. As illustrated, the receiver 102, the pedicle screw 104, and the rod 106 may be assembled to form the spinal alignment system 100.

As illustrated in FIGS. 1A-1C, the receiver 102 may include a first outside portion 108, having a first lower potion 108a and a first upper portion 108b, and including a first pair of legs 109, of which first leg 109a and second leg 109b are shown. Also shown is a second inside portion 110, having a second lower portion 110a and a second upper portion 110b, and including a second pair of legs 111 of which third leg 111a and fourth leg 111b are shown, one or more tabs 112 of which first tab 112a and second tab 112b are shown. Also shown are the first tab 112a disposed on an inner surface of the first outside portion 108 at or at about the first pair of legs 109, and the second tab 112b disposed on an inner surface of the second inside portion 110 at or at about the second pair of legs 111. The first outside portion 108 and the second inside portion 110 may be coupled to each other by way of a plurality of hinged connections 116 of which first and second hinged connections 116a and 116b, respectively, are shown. Although FIGS. 1A and 1B illustrate that the receiver 102 includes two pair of legs (i.e., the first and second pair of legs 109 and 111), it will be apparent to a person skilled in the art that the scope of the present disclosure is not limited to such a construction. In various other aspects, the receiver 102 may include any number of pairs of legs without deviating from the scope of the present disclosure. In such a scenario, each pair of legs is configured to perform one or more operations in a manner similar to the operations of the first pair of legs 109 and second pairs of legs 111 as described herein. In some embodiments of the present disclosure, the plurality of hinged connections 116 may include a plurality of wire springs 118 of which first and second wire springs 118a and 118b are shown and a plurality of pivot pins 120 of which first and second pivot pins 120a and 120b are shown. Specifically, the first hinged connection 116a that may be adapted to couple the first leg 109a and the third leg 111a may include the first wire spring 118a and the first pivot pin 120a. Similarly, the second hinged connection 116b that may be adapted to couple the second leg 109b and the second leg 111b may include the second wire spring 118b and the second pivot pin 120b.

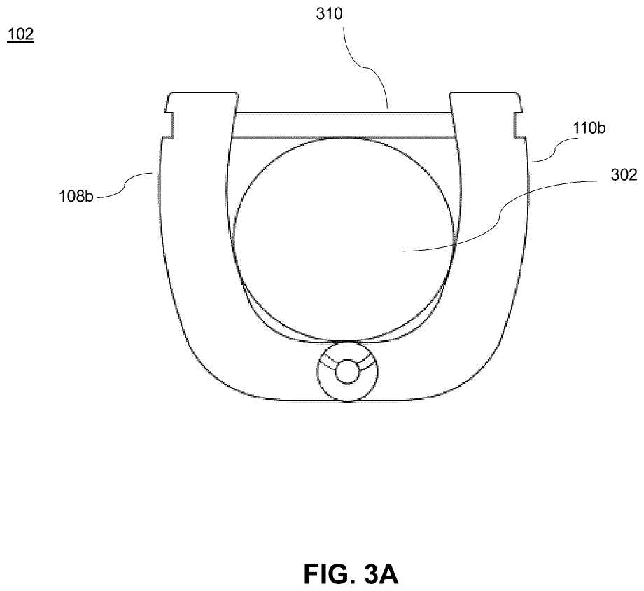

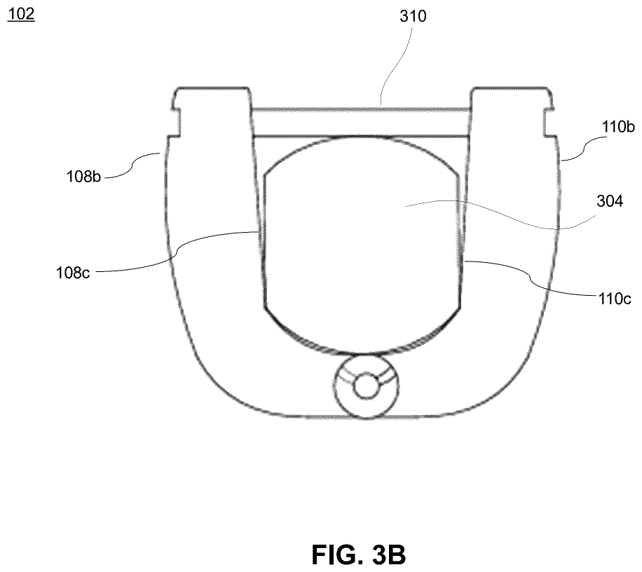

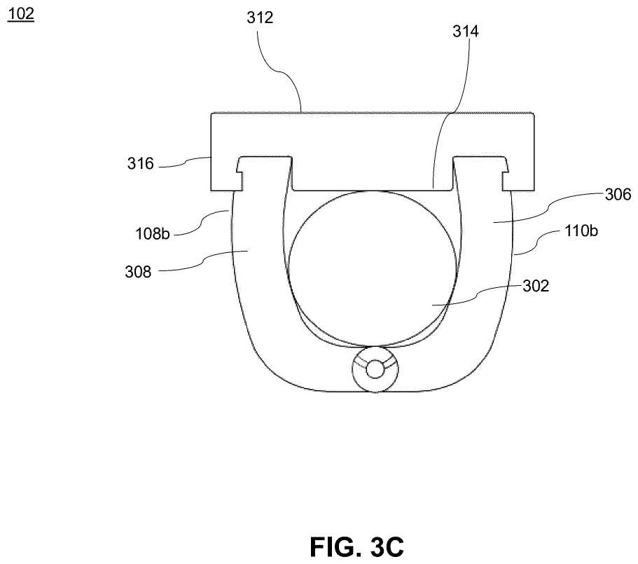

The plurality of hinged connections 116 may enable the first outside portion 108 and the second inside portion 110 to exhibit one of, a neutral or aligned position or an angled position relative to each other. Specifically, in this embodiment, in the neutral or aligned position, the first outside portion 108 and the second inside portion 110 are at a substantially parallel position with respect to one another. Specifically, the first outside portion 108 and the second inside portion 110 in the neutral or aligned position defines a first opening 122a, a second opening 122b, a third opening 122c, and a fourth opening 122d. In some embodiments of the present disclosure, as shown in FIG. 1C, the receiver 102 may have a distal face 102a, a central portion 102b, and a proximal face 102c such that the first opening 122a is defined at the distal face 102a, the second opening 122b and the third opening 122c are defined at the central portion 102b, and the fourth opening 122d is defined at the proximal face 102c. In certain embodiments, a retaining device 114 may be used to prevent outward or inward movement of the outside portion 108 or the inside portion 110. The retaining device may be positioned after insertion of a rod through the fourth opening. The retaining device may be in the form of a rod, a plate, a chain, a spring, a cap, a cover, a lid, a ring, a c-ring, a clip, or any other rigid material. Alternatively, the retaining device 114 may be positioned after a plug (see FIGS. 3A and 3B, item 310) is positioned between the first upper portion 108b and the second upper portion 110b.

In the contracted position, the first upper portion 108b of the first outside portion 108 and the second upper portion 110b of the second inside portion 110 are forced inwards towards each other. Specifically, when the first upper portion 108b of the first outside portion 108 and the second upper portion 110b of the second inside portion 110 are contracted or compressed by applying an external force on the first upper portion 108b and the second upper portion 110b such that the first upper portion 108b and the second upper portion 110b are moved towards each other to exhibit the contracted or compressed position, the first opening 122a is expanded. In some embodiments of the present disclosure, the first opening 122a, when expanded by contracting the first upper portion 108b and the second upper portion 110b, accepts the head 104b of the pedicle screw 104. The contraction of the first upper portion 108b and the second upper portion 110b to expand the first opening 122a may be enabled by way of the plurality of hinged connections 116 attaching the first and second pair of legs 109 and 111.

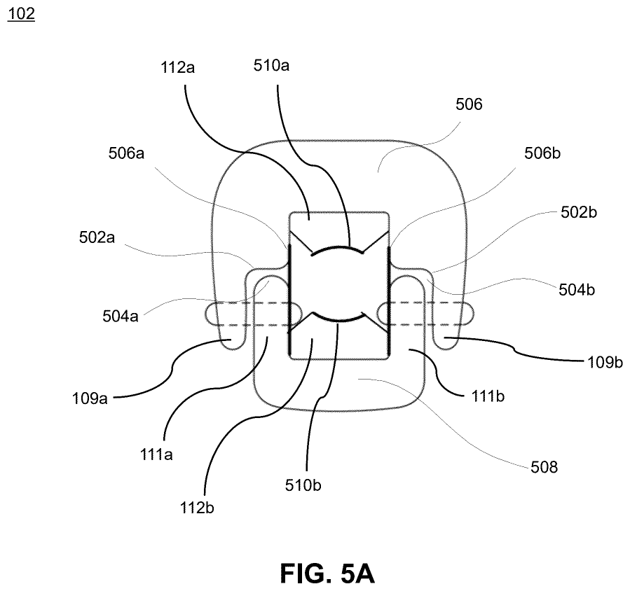

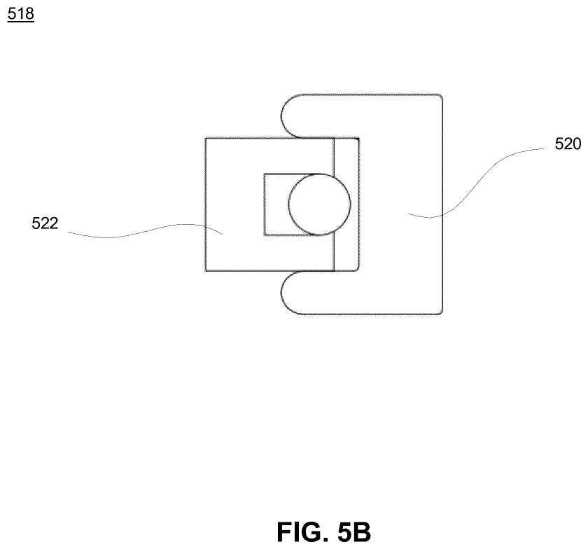

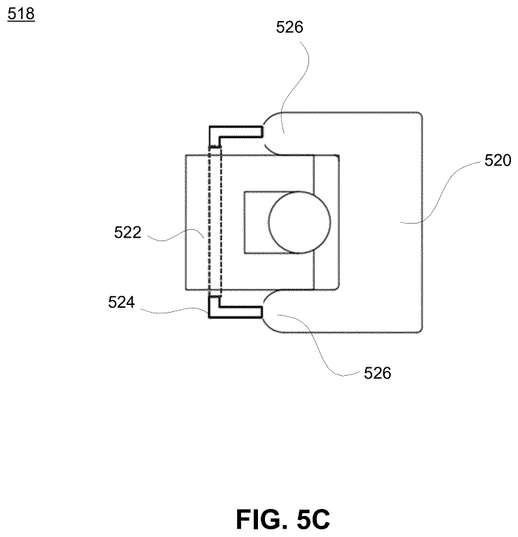

In certain embodiments, the first upper portion 108b and the second upper portion 110b may be prevented from moving outward away from each other. This can be accomplished for example, as shown in FIG. 5C, where bar 524 extends from the first pair of legs 526 and below the second inside portion 522, such that the bar is forced upward against the bottom of the second inside portion 522 when an attempt is made to force apart the first upper portion 108b and second upper portion 110b, thereby preventing such outward movement. In one or more embodiments, there are no flanges on the first upper portion 108b and the second upper portion 110b. In one or more embodiments flanges are includes and the bar 524 is positioned such that there is a slight outward movement allowed for each of the first upper portion 108b and the second upper portion 110b. This allows insertion of a rod past the flanges for positioning within the receiver 518.

In certain embodiments, the first upper portion 108b of the first outside portion 108 and the second upper portion 110b of the second inside portion 110 are forced outwards away from each other. Specifically, when the first upper portion 108b of the first outside portion 108 and the second upper portion 110b of the second inside portion 110 are expanded or separated by applying an external force on the first upper portion 108b and the second upper portion 110b such that the first upper portion 108b and the second upper portion 110b are moved apart from each other to exhibit the expanded or separated position, the first opening 122a is expanded. In some embodiments of the present disclosure, the first opening 122a, when expanded by expanding or separating the first upper portion 108b and the second upper portion 110b, accepts the head 104b of the pedicle screw 104. The separation of the first upper portion 108b and the second upper portion 110b to expand the first opening 122a may be enabled by way of the plurality of hinged connections 116 attaching the first and second pair of legs 109 and 111.

In some embodiments of the present disclosure, each leg of the first and second pair of legs 109 and 111 may be positioned horizontally with respect to a vertically positioned first lower portion 108a and second lower portion 110a. Specifically, the first leg 109a of the first pair of legs 109 may be positioned horizontally. Similarly, the second leg 109b of the first pair of legs 109 may be positioned horizontally. Similarly, the third leg 111a of the second pair of legs 111 may be positioned horizontally. Similarly, the fourth leg 111b of the second pair of legs 111 may be positioned horizontally. Further, each leg of the first pair of legs 109 and second pair of legs 111 may include at least one through hole 124a-124d. Specifically, each of the horizontally positioned legs 109a, 109b, 111a, and 111b may include the at least one through hole 124a-124d, respectively. In some embodiments of the present disclosure, to couple the first pair of legs 109 to the second pair of legs 111, the at least one through hole 124a and 124b of the first pair of legs 109 is aligned with the at least one through hole 124c and 124d of the second pair of legs 111 such that the at least one through hole 124a-124d receives a wire spring (the first and second wire springs 118a and 118b) of the plurality of wire springs 118 and a pivot pin (the first and second pivot pins 120a and 120b) of the plurality of pivot pins 120. In some embodiments of the present disclosure, the horizontally positioned legs 109a, 109b, 111a, and 111b and the vertically positioned first lower portion 108a and second lower portion 110a may be integrally coupled. Embodiments of the present disclosure are intended to include or otherwise cover any type of coupling to couple the horizontally positioned legs 109a and 109b to the first lower portion, and the horizontally positioned legs 111a and 111b to the second lower portion 111a and 111b, without deviating from the scope of the present disclosure.

The one or more first tabs 112a may be disposed onto an inner surface of the first outside portion 108 and the one or more second tabs 112b may be disposed onto an inner surface of the first inside portion 110. Specifically, the one or more first tabs 112a may be disposed at the first lower portion 108a on the inner surface just above, just below (or anywhere in between) the first pair of legs 109 and the one or more second tabs 112b may be disposed at the second lower portion 110a on the inner surface at about or just above the second pair of legs 111, respectively. One or more third tabs 112c and fourth tabs 112d may be provided and adapted to engage the rod 106 after the rod 106 is inserted through the fourth opening 122d when the first outside portion 108 and the second outside portion 110 may be in the neutral position. As illustrated in FIG. 1B, the one or more tabs 112c and 112d may be a protrusion extending outwards from the first inner surfaces of the first outside portion 108 and the second inner surface of the second inside portion 110. In some embodiments of the present disclosure, the one or more tabs 112c and 112d may be telescopic tabs that pop out from the inner surfaces of the first outside portion 108 and the second inside portion 110 when the rod 106 is inserted into the fourth opening 122d and is to be retained with the second opening 122b, the third opening 122c and the fourth opening 122d. Embodiments of the present disclosure are intended to include and/or otherwise cover any type of the one or more tabs 112a and 112b that may enable efficient engagement and retention of the rod 106 within the second opening 122b, the third opening 122c, and the fourth opening 122d, without deviating from the scope of the present disclosure. For example, the tabs 112a and 112b may be configured with an upper surface that slopes downward and inward and a bottom surface that is horizontal and flat, thereby making it easier to insert rod 106 but more difficult to remove rod 106 from the fourth opening 122d.

The retaining means 114 may be adapted to couple the first upper portion 108b with the second upper portion 110b. Specifically, the retaining means 114 may be adapted to couple the first upper portion 108b with the second upper portion 110b to retain the first upper portion 108b and the second upper portion 110b in the neutral position. In some embodiments of the present disclosure, the retaining means 114 may be a wire spring, a rod, a plate, a chain, a spring, a cap, a cover, a lid, a ring, a c-ring, a clip, or any other rigid material fixedly attached to the first upper portion 108b and the second upper portion 110b to retain the first upper portion 108b and the second upper portion 110b in the neutral position. In some embodiments of the present disclosure, the retaining means 114 may be a wire spring, a rod, a plate, a chain, a spring, a cap, a cover, a lid, a ring, a c-ring, a clip, or any other rigid material removably attached to the first upper portion 108b and the second upper portion 110b to retain the first upper portion 108b and the second upper portion 110b in the neutral position. Embodiments of the present disclosure are intended include or otherwise cover any other retaining means 114 that is capable of retaining the neutral position of the first upper portion 108b and the second upper portion 110b when the external force applied on the first upper portion 108b and the second upper portion 110b is released, without deviating from the scope of the present disclosure.

The pedicle screw 104 may include a threaded portion 104a and a head portion 104b. The head portion 104b may be coupled to the threaded portion 104a. The threaded portion 104a may be adapted for securing the pedicle screw 104 to a vertebra. The head portion 104b may be adapted to be inserted into the first opening 122a when the first upper portion 108b and the second upper portion 110b are in the second position, which may be a contracted position or an expanded position (i.e., the first opening 122a is expanded) to couple the pedicle screw with the receiver 102. In other words, when the external force applied on the first upper portion 108b and the second upper portion 110b is released, the first outside portion 108 and the second inside portion 110 return to the first or neutral position and thereby holds the head portion 104b of the pedicle screw 104 within the first opening 122a.

The rod 106 may be adapted to be inserted through the fourth opening 122d. Specifically, the rod 106 may be adapted to be inserted through the fourth opening 122d such that the rod 106 is engaged by way of the one or more tabs 112c and 112d when the first upper portion 108b and the second upper portion 110b are in the first or neutral position. In some embodiment of the present disclosure, the rod 106 may include, but is not limited to, one of a cylindrical rod, a rod with one or more flat sides, a tapered rod, or a rod having a trapezoidal cross section. Embodiments of the present disclosure are intended to include or otherwise cover any type of the rod 106, without deviating from the scope of the disclosure.

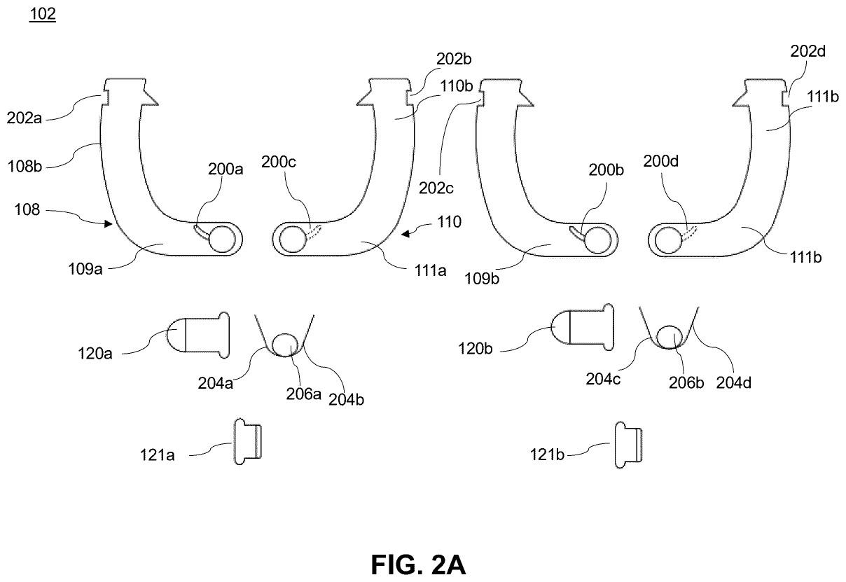

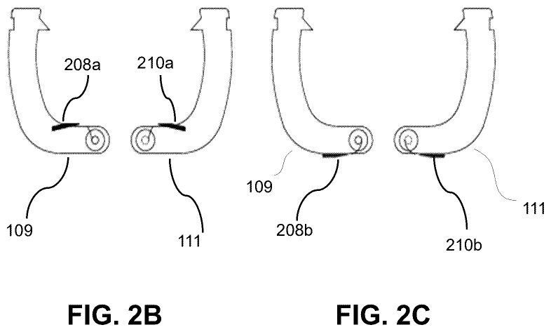

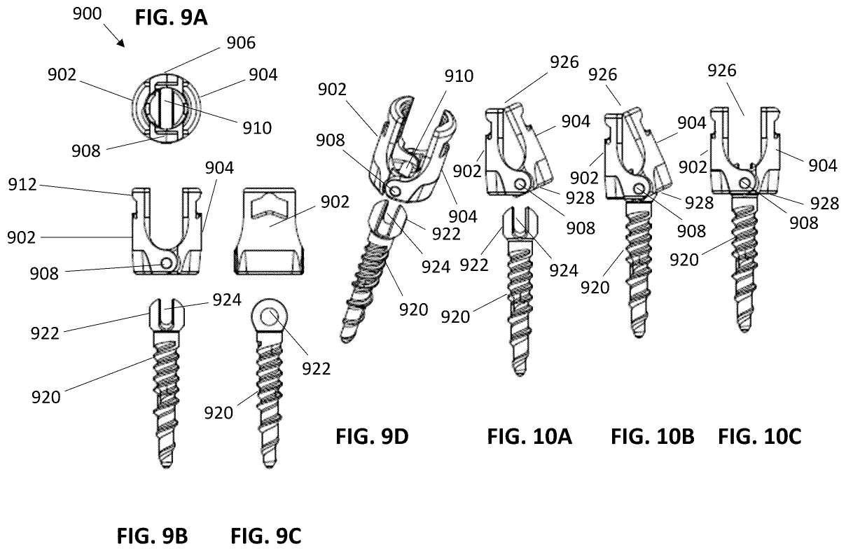

FIG. 2A illustrates an exploded side view of receiver 102 of the spinal alignment system 100 of FIGS. 1A-1C, according to an embodiment of the present disclosure. As discussed, the receiver 102 may include the first pair of legs 109, the second pair of legs 111, the one or more first tab 112a and second tab 112b (as depicted in FIGS. 9A and 9B), the plurality of wire springs 118 (hereinafter individually referred to and designated as “the wire spring 118” and collectively referred to and designated as “the wire springs 118”, as depicted in FIG. 1A), the plurality of pivot pins 120a and 120b (hereinafter individually referred to and designated as “the pivot pin 120a” and “the pivot pin 120b” and collectively referred to and designated as “the pivot pins 120”). In some embodiments of the present disclosure, the first horizontal portions of the first leg 109a of the first pair of legs 109 and of the second leg 109b of the first pair of legs comprising first and second slot 200a and 200b, respectively, on an outer surface or inner surface. Similarly, the horizontal portions of the third leg 111a and of the fourth leg 111b of the second pair of legs 111 may include third and fourth slots 200c and 200d, respectively, on an outer surface or inner surface.

In some embodiment of the present disclosure, the first upper portion 108b of the first outside portion 108 comprising a first horizontal slot or groove 202a on the outer surface. Similarly, the second upper portion 110b of the second inside portion 110 may include a second horizontal slot or groove 202b on the outer surface. The horizontal slots 202a-202b may facilitate accepting one or more rings, caps, covers, lids, c-rings, clips, or tools therein. Alternatively, the receiver may include more than two horizontal slots, such as for example, 3, 4, 5, 6, 7, 8, 9, or 10 horizontal slots or holes in the outer surface of the first upper portion 108b of the first outside portion 108 and the second upper portion 110b of the second inside portion 110. The one or more tools may include, but not limited to, the tools that are required during surgical operation of spine of a subject to implant the spine alignment system 100 of the present disclosure. Embodiments of the present disclosure are intended to include or otherwise cover, other types of tools including known, related and later developed tools, without deviating from the scope of the present disclosure. The one or more rings, caps, covers, lids, c-rings, and clips, may be used to retain the first outside portion 108 and the second inside portion 110 in the first or neutral position, thereby causing retention of the head 104b of the pedicle screw (not shown) by the receiver. They may also be used to cause retention of the rod within the second opening, the third opening, and the fourth opening, and preventing the exiting of the rod from the receiver.

As shown in FIG. 1A, the plurality of hinged connections 116 may include a plurality of wire springs 118 of which first and second wire springs 118a and 118b are shown and a plurality of pivot pins 120 of which first and second pivot pins 120a and 120b are shown, respectively. Specifically, the first hinged connection 116a may include the first wire spring 118a and the first pivot pin 120a. Similarly, the second hinged connection 116b may include the second wire spring 118b and the second pivot pin 120b. In some embodiments of the present disclosure, to couple the first pair of legs 109 with the second pair of legs 111, the at least one through hole 124a of the first pair of legs 109 may be aligned with the at least one through hole 124c of the second pair of legs 111 and the at least one through hole 124b of the first pair of legs 109 may be aligned with the at least one through hole 124d of the second pair of legs 111 such that the at least one through holes 124a and 124c receive the first wire spring 118a and the first pivot pin 120a and the at least one through holes 124b and 124d receive the second wire spring 118b, and the second pivot pin 120b.

In some embodiments of the present disclosure, each wire spring of the plurality of wire springs 118 may include a plurality of upright portions 204 and a central portion 206. Specifically, the first wire spring 118a may include upright portions 204a and 204b and the central portion 206a. Similarly, the second wire spring 118b may include the upright portions 204c and 204d and the central portion 206b. In some embodiments of the present disclosure, the plurality of upright portions 204a-204d of the first and second wire spring 118a and 118b may be engaged within the first to fourth slots 200a-200d, respectively, provided on the outer surface or inner surface of the first pair of legs 109 and on the inner surface or outer surface of the second pair of legs 111 when the first and second pair of legs 109 and 111 are aligned. In some embodiments of the present disclosure, the first and second wire springs 118a-118b may facilitate in exerting a spring force on the first pair of legs 109 and the second pair of legs 111 when the first outside portion 108 and the second inside portion 110 are in the second position, inwardly leaning towards or outwardly leaning away from each other (i.e., in a contracted or extended position), thereby the first and second wire springs 118a-118b may tend to keep the first pair of legs 108 and the second pair of legs 110 in the first or neutral position. Although FIG. 2A illustrates that the receiver 102 includes a single pair of wire springs (i.e., the first and second wire springs 118a-118b), it will be apparent to a person skilled in the art that the scope of the present disclosure is not limited to such construction. In various other embodiment, the receiver 102 may include only a single wire spring or multiple wire springs similar to the first and second wire springs 118a-118b, without deviating from the scope of the present disclosure.

The plurality of pivot pins 120 (i.e., the first and second pivot pins 120a and 120b) may be adapted to facilitate in the hinged connection of the first pair of legs 108 and the second pair of legs 110. In some embodiments of the present disclosure, the plurality of pivot pins 120 (i.e., the first and second pivot pins 120a and 120b) may include a rough surface that may facilitate a frictionally fit hinged connection of the first pair of legs 109 and the second pair of legs 111. Although FIG. 2A illustrates that the receiver 102 includes a single pair of pivot pins (i.e., first and second pivot pins 120a and 120b), it will be apparent to a person skilled in the art that the scope of the present disclosure is not limited to such construction. In various other embodiment, the receiver 102 may include any number of pivot pins, without deviating from the scope of the present disclosure. In some embodiments of the present disclosure, the plurality of pivot pins 120 may be adapted to facilitate in hinged connection of the first pair of legs 109 and the second pair of legs 111 when the at least one through hole 124a of the first pair of legs 109 may be aligned with the at least one through hole 124c of the second pair of legs 111 and the at least one through hole 124b of the first pair of legs 109 may be aligned with the at least one through hole 124d of the second pair of legs 111, such that the at least one through hole 124a receives the first wire spring 118a and the first pivot pin 120a and the at least one through hole 124b receives the second wire spring 118b and the second pivot pin 120b.

In certain embodiments, as shown in FIG. 2A, the pivot pin 102a may include a first cap 121a that couples to the first pin 120a to lock the pin 120a in place or prevent the pin from coming loose. Pivot pin 120b may include a second cap 121b that couples to the second pin 120b to lock the pin 120b in place. Caps 121a and 121b may be coupled to the respective pin 120a or 120b, via a snap fit, a screw fit, a pressure fit, adhesive, friction fit, surface roughening, welding, bonding, or some other method.

FIG. 2B illustrates certain embodiments, where the first tab 208a is positioned above the first pair of legs 109 and the second tab 210a is positioned above the second pair of legs 111.

FIG. 2C illustrates certain embodiments, where the first tab 208b is positioned below the first pair of legs 109 and the second tab 210b is positioned below the second pair of legs 111. However, the first tab 208b may be located at any position between the first pair of legs 109 and the second tab 210b may be located at any position between the second pair of legs 111.

FIGS. 3A-3C illustrate a side view of the receiver 102 with the rod 106, according to multiple embodiments of the present disclosure. As illustrated in FIG. 3A, the receiver 102 may be adapted to receive a cylindrical rod 302. In other words, the receiver 102 may be adapted to receive a rod 302 having circular cross section. Specifically, the receiver 102 may be adapted to receive the cylindrical rod 302 when the first pair of legs 109 and the second pair of legs 111 of the receiver 102 may be aligned and coupled to each other by way of the plurality of hinged connections 116 such that the first outside portion 108 and the second inside portion 110 are in the first position and exhibit the neutral position.

In certain embodiments, as shown in FIG. 3A, where the first upper portion 108b and the second upper portion 110b flex inward for insertion of the pedicle screw 104, the rod 302 is held in place using a plug 310 that is positioned in the space between the first upper portion 108b and the second upper portion 110b and above the rod. The plug 310 may be cylindrically shaped and include threaded sidewalls which will help retain the plug 310 in position using matching threads on the inside walls of the first upper portion 108b and the second upper portion 110b. Alternatively, the plug 310 may be retained in place using a gasket with a pressure fit, snap fit, adhesive, friction fit, surface roughening, welding, bonding, or some other method.

In FIG. 3B, in place of a rod 302 with a circular cross section, a flat sided rod 304 is used. In this configuration, the first upper portion 108b and the second upper portion 110b each include a flat inner side wall 108c, 110c.

In certain embodiments, as shown in FIG. 3C, where the first upper portion 108b and the second upper portion 110b flex outward for insertion of the pedicle screw 104, the rod 302 is held in place using a plug and cap 312 that is positioned in the space between the first upper portion 108b and the second upper portion 110b and above the rod, as well as extending around the peripheries of the first upper portion 108b and the second upper portion 110b. The plug and cap 312 may be cylindrically shaped and include threaded sidewalls on the periphery of the plug portion 314 and on the inside edge of the cap portion 316 which will help retain the cap 310 in position using matching threads, respectively, on the inside walls and outside walls of the first upper portion 108b and the second upper portion 110b. Alternatively, the plug and cap 312 may be retained in place using one or more gaskets with a pressure fit, using a snap fit, an adhesive, a friction fit, a surface roughening, a welding, a bonding, or some other method.

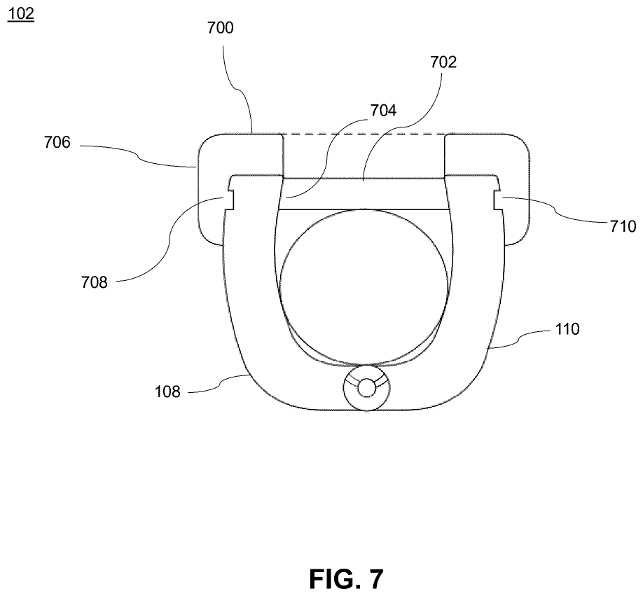

Alternatively, the plug portion 314 and the cap portion 316 may be separate elements, with the plug portion 314 being positioned first to retain the rod in a fixed position relative to the pedicle screw 104. Thereafter, the cap portion 316 may be positioned to retain the plug portion 314 in a fixed position. Alternatively, as shown in FIG. 7, the cap portion 316 may include an aperture 320 through which the plug portion 314 may be inserted after the cap portion 316 is positioned relative to the first upper portion 108b and the second upper portion 110b.

As illustrated in FIG. 3B, the receiver 102 with the rod 304 is structurally and functionally similar to the receiver 102 with the rod as illustrated in FIG. 3A. However, the receiver 102 (as illustrated in FIG. 3B) may include another rod 304 having one or more flat sides. In other words, the receiver 102 may be adapted to receive a rod 304 having a circular cross section with one or more flat sides. The one or more flat sides of the rod 304 may facilitate in appropriate gripping of the rod 304 with the inner surfaces of the first outside portion 108 and the second inside portion 110 when the first pair of legs 109 and the second pair of legs 111 of the receiver 102 may be aligned and coupled to each other by way of the plurality of hinged connections 116 such that the first outside portion 108 and the second inside portion 111 exhibit the first or neutral position.

As illustrated in FIG. 3C, the receiver 102 with the rod 302 is structurally and functionally similar to the receiver 102 with the rod as illustrated in FIGS. 3A and 3B. However, the receiver 102 (as illustrated in FIG. 3C) may include a first outside portion 306 and a second inside portion 308 without including any tabs to engage the rod.

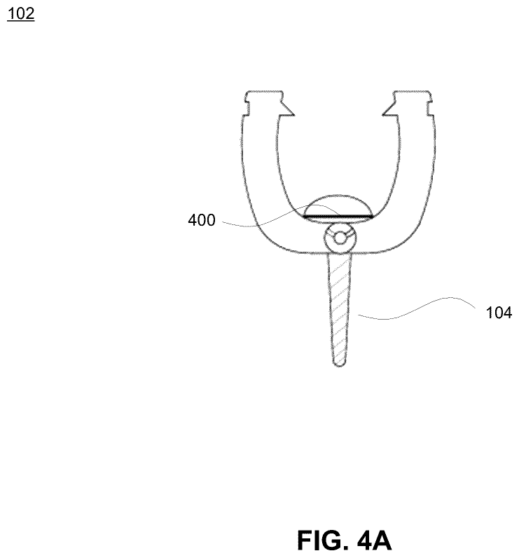

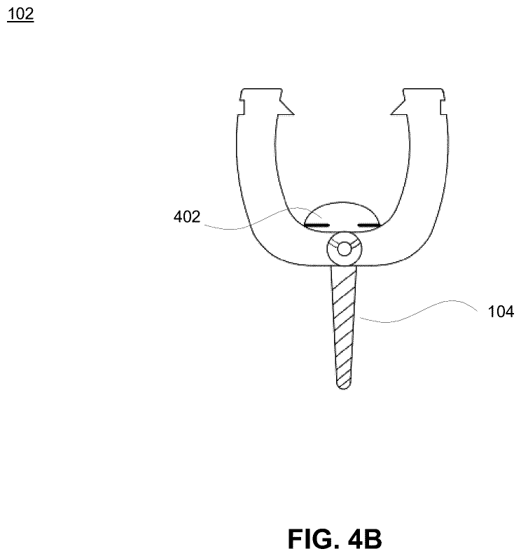

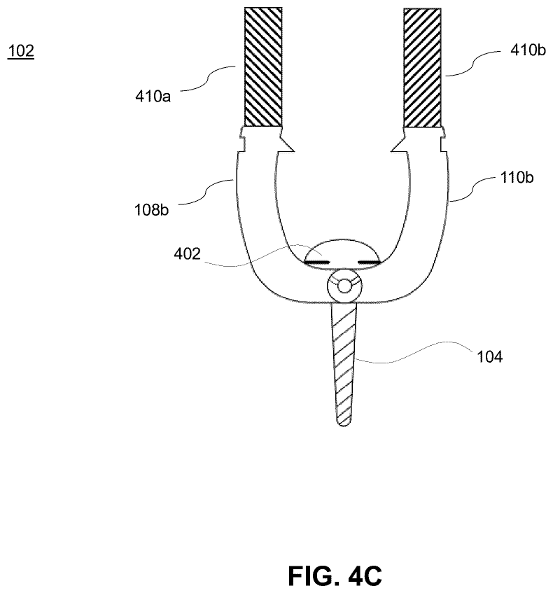

FIGS. 4A-4B illustrate a side view of the receiver 102 with the pedicle screw 104, and without a rod in place, according to an embodiment of the present disclosure. As illustrated in FIG. 4A, the threaded portion 104b of the pedicle screw 104 may be adapted to be inserted through a locking washer 400. Specifically, the threaded portion 104b of the pedicle screw 104 may be adapted to be inserted through the locking washer 400 such that the locking washer 400 may rest proximate to the head portion 104b of the pedicle screw 104 when the receiver 102 may be attached to the pedicle screw 104. The locking washer 400 may facilitate in preventing looseness of the pedicle screw 104 when the pedicle screw 104 may be received in the first opening 122a of the receiver 102.

As illustrated in FIG. 4B, the threaded portion 104b of the pedicle screw 104 may be adapted to be inserted through a c-ring 402. Specifically, the threaded portion 104b of the pedicle screw 104 may be adapted to be inserted through the c-ring 402 such that the c-ring 402 may rest proximate to the head portion 104b of the pedicle screw 104 when the receiver 102 may be attached to the pedicle screw 104. The c-ring 402 may facilitate in preventing looseness of the pedicle screw 104 when the pedicle screw 104 may be received in the first opening 122a of the receiver 102

As illustrated in FIG. 4C, there is shown a side view of the receiver 102 with the pedicle screw 104, and without a rod in place, according to an embodiment of the present disclosure. Removably coupled to the top of each of the first upper portion 108b and the second upper portion 110b is an extension arm 410a, 410b. The extension arms are used to provide greater surface and grip area for compression of the first upper portion 108b and the second upper portion 110b to provide for insertion of the pedicle screw. Alternatively, the extension arms may be coupled to the outside, inside, or over the tops of the first upper portion 108b and the second upper portion 110b. The extension arms 410a, 410b may be coupled using adhesive or bonding, or they can be unitary with each of the first upper portion 108b and the second upper portion 110b, with a weakened connection point that would allow for easy removal after the receiver 102 is positioned with respect to the pedicle screw. Any other known or future discovered connection method may be used to couple the extension arms 410a, 410b to the first upper portion 108b and the second upper portion 110b, respectively. The extension arms 410a, 410b may be formed of plastic, wood, metal, ceramic, an alloy, bone, or any other rigid material that could withstand the counter pressure applied to the first pair of legs 109 and the second pair of legs 111 by the springs 118a, 118b.

FIG. 5A illustrates a top view of the receiver 102, according to an embodiment of the present disclosure. As illustrated in FIG. 5A, the first pair of legs 109 and the second pair of legs 111 of the receiver 102 may be integrally connected to form the outside portion in the form of a U-shaped structure 506 and the inside portion in the form of a U-shaped structure 508, respectively. In some embodiments of the present disclosure, the U-shaped structure 506 is bigger than the U-shaped structure 508 thereby enabling the second pair of legs 111 to be positioned inside the first pair of legs 109. In some embodiment of the present disclosure, each of the first leg 109a and the second leg 109b of the first pair of legs 109 may include a groove portion 502a and 502b, respectively. Further, each of the third leg 111a and fourth leg 111b of the second pair of legs 111 may include a curved portion 504a and 504b, respectively, disposed at a tip portion of each of the third leg 111a and the fourth leg 111b, respectively. When the at least one through hole 124a of the first leg 109a of the first pair of legs and the at least one through hole 124c of the third leg 111a of the second pair of legs is aligned and the at least one through hole 124b of the second leg 109b of the first pair of legs and the at least one through hole 124d of the fourth leg 111b of the second pair of legs, respectively are aligned, then the groove portion 502a may be adapted to receive the curved portion 504a and the grooved portion 502b may be adapted to receive the curved portion 504b.

In some embodiment of the present disclosure, the first tab 112a and the second tab 112b may form a pair of upward facing lips 510a, 510b at a bottom of the receiver 102 when the first and the second pair of legs 108,110 exhibits the neutral position. The pair of upward facing lips 510a, 510b may be adapted to receive downward facing overhang of a mushroom shaped head portion of the pedicle screw 104. Specifically, the pair of upward facing lips 510a, 510b may be adapted to receive the downward facing overhang of the mushroom shaped head portion of the pedicle screw 104 when the mushroom shaped head portion of the pedicle screw 104 may be adapted to be inserted into the first opening 122a (i.e., when the first outside portion 108 and the second inside portion 110 are in the second, contracted or expanded position).

FIG. 5B illustrates a yet another top view of another receiver 518, according to an embodiment of the present disclosure. The receiver 518 may include a first pair of legs 520 and a second pair of legs 522. As illustrated in FIG. 5B, the receiver 518 is structurally and functionally similar to the receiver 102 of FIG. 5A. However, in the receiver 518 of FIG. 5B, each horizontal portion of the first pair of legs 520 and each horizontal portion of the second pair of leg 522 does not include the groove portion and curved portion (as illustrated in FIG. 5A).

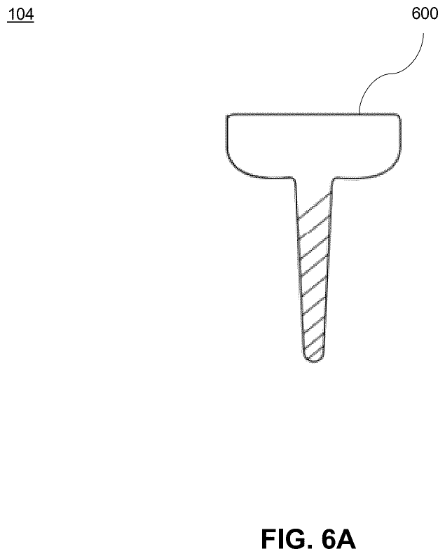

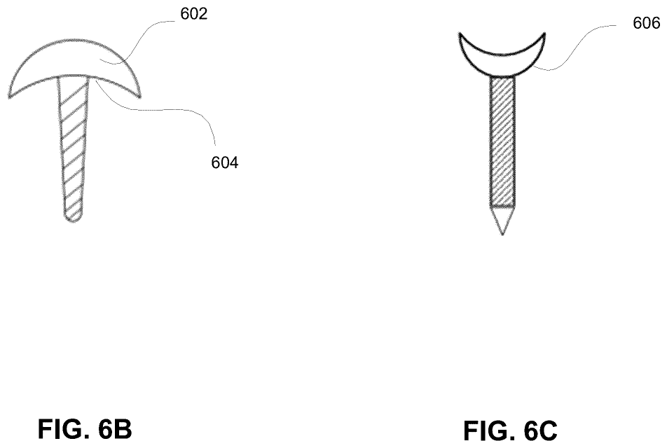

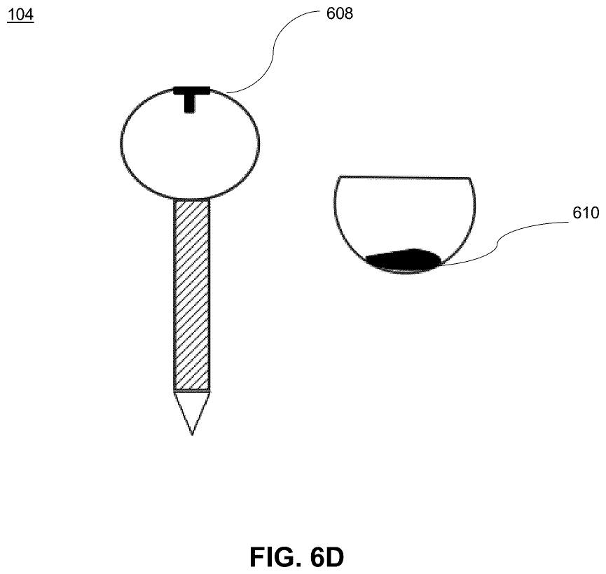

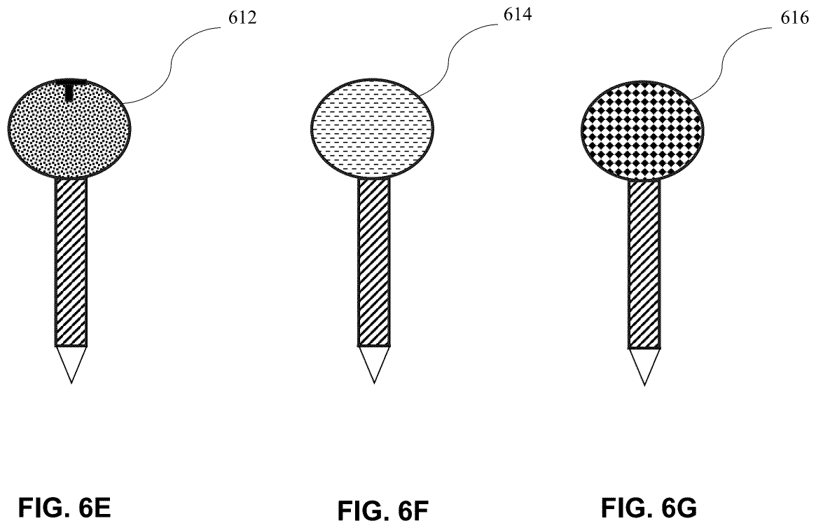

FIG. 6A illustrates the pedicle screw 104 with a head portion 600 having a flat surface, according to an embodiment of the present disclosure. As illustrated, the flat surface of the head portion 600 may facilitate in reduction of height of the pedicle screw. Further, the flat surface of the head portion 600 may further increase space inside the second opening 122b when the first outside portion 108 and second inside portion 110 exhibit the first or neutral position thereby facilitating in receiving a rod 106 having a larger diameter.

FIG. 6B illustrates a pedicle screw 104 with another head portion 602 having a mushroom shape, according to an embodiment of the present disclosure. As illustrated the head portion 602 in the mushroom shape may include a downward facing overhang 604 that may be adapted to engage with the pair of upward facing lips 506a, 506b of the receiver 102. The engagement of the downward facing overhang 604 with the pair of upward facing lips 506a, 506b, results in the pedicle screw 104 being tightly secured in the first opening 122a.

In certain embodiments, as shown in FIG. 6C, the head portion 606 of the pedicle screw 104 can have a concave shape.

FIG. 6D illustrates a pedicle screw 104 with a head portion 608 having a spherical shape, according to an embodiment of the present disclosure. Also shown is a collar 610 through which and into which the pedicle screw 104 is inserted and which retains the head portion 608 and provides for rotational movement of the pedicle screw 104.

As shown in FIGS. 6E, 6F and 6G, the head portions 612, 614, and 616, respectively illustrate various embodiments that include surface structures that provide for retention of the pedicle screw 104 is a fixed position upon application of pressure between the respective head portion 612, 614, 616 and the collar 610. FIG. 6E shows a roughened surface for the head portion 612. FIG. 6F shows a head portion 614 including a series of ridges. FIG. 6G shows the head portion 616 including a series of notches. The surface structures can include other features, including ridges, grooves, notches, indentations, lines, waves, furrows, scratches, roughened surfaces, or other artifacts that provide friction. These surface structures, in combination with each corresponding collar 610, which may include surface structures on the inside portion of the collar 610 that corresponds to the surface structures on the corresponding head portion 612, 614, 616, provide for retention of the pedicle screw 104 in various fixed positions relative to the retaining element 102.

FIG. 7 illustrates a side view of the receiver 102 with a cap 700, according to an embodiment of the present disclosure. As illustrated in FIG. 7, the cap 700 may include an open center portion. In some embodiment of the present disclosure, the cap 700 may be attached to the proximal face 122c of the receiver 102. In some embodiment of the present disclosure, the inner portion of the cap 702 may have an outer edge 704 and the outer portion of the cap 706 may have an inner edge 708 to prevent the first outside portion 108 and the second inside portion 110 from moving when the first outside portion 108 and second inside portion 110 exhibit the first or neutral position. In some other embodiments of the present disclosure, the cap may be adapted to be received over a nipple or an edge portion 710 on the first outside portion 108 and the second inside portion 110 such that the cap 700 may snap fit to secure the first upper portion 108b and the second upper portion 110b when the first outside portion 108 and second inside portion 110 exhibit the neutral position.

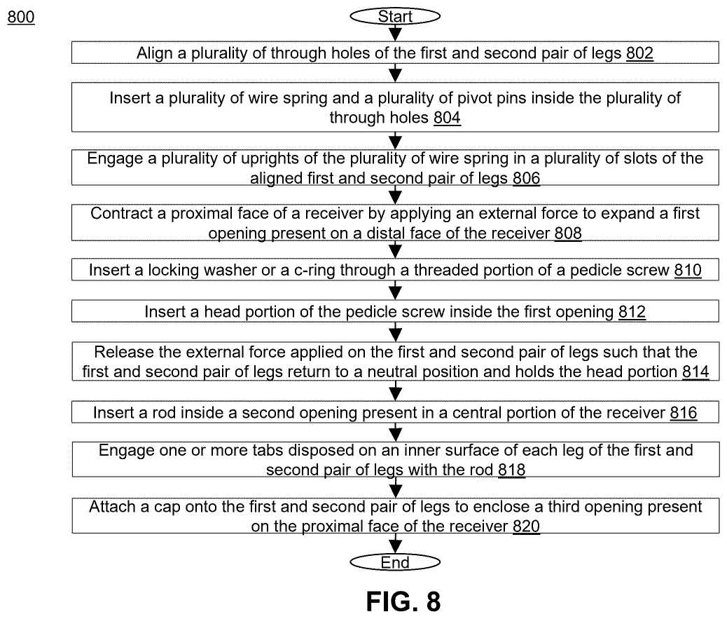

FIG. 8 illustrates a flowchart of a method 800 for assembling the spinal alignment system 100, according to an embodiment of the present disclosure.

At step 802, a user may align the plurality of through holes of the first pair of legs 109 and the second pair of legs 111. Specifically, to align the first pair of legs 109 and the second pair of legs 111, a through hole 124a of a first leg 109a of the first pair of legs 109 is aligned with a thorough hole 124c of a third leg 111a of the second pair of legs 111. Similarly, a through hole 124b of a second leg 109b of the first pair of legs 109 is aligned with a thorough hole 124d of a fourth leg 111b of the second pair of legs 111.

At step 804, the user may insert the plurality of wire springs 118a, 118b and the plurality of pivot pins 120a, 120b inside the plurality of through holes 124.

At step 806, the user may engage the plurality of uprights 204a, 204b, 204c, 204d of the plurality of wire springs 118a, 118b in the plurality of slots 124a, 124b, 124c, 124d of the aligned first and second pair of legs 109 and 111.

At step 808, the user may contract or compress a proximal face of the receiver 102 at the first upper portion 108b and the second upper portion 110b by applying an external force on the first upper portion 108b and the second upper portion 110b of the receiver 102 such that the the first upper portion 108b and the second upper portion 110b are forced towards each other to exhibit a contracted position or away from each other to form an expanded position thus expanding a first opening 102a present at a distal face of the receiver 102. The contraction or expansion is enabled by way of a plurality of hinged connections coupling the first pair of legs 109 and the second pair of legs 111.

At step 810, the user may insert a locking washer 400 or a c-ring 402 through a threaded portion 104a of the pedicle screw 104.

At step 812, the user may insert a head portion 600, 602 of the pedicle screw 104 inside the first opening 102a. Specifically, the head portion 600, 602 of the pedicle screw 104 is inserted inside the expanded first opening formed by virtue of the contraction of the first pair of legs 109 and the second pair of legs 111.

At step 814, the user may release the external force applied on the first upper portion 108b and on the second upper portion 111b such that the first upper portion 108b and the second upper portion 111b return to a neutral position and the tabs 112a, 112b retain the head portion 600, 602 securely in the retainer.

At step 816, the user may insert a rod through a fourth opening 102d present at a proximal face of the receiver 102. Specifically, the fourth opening 102d is formed by the structure of the first upper portion 108b and the second upper portion 110b when the receiver 102 is in the neutral or an expanded position.

At step 818, the user may engage one or more tabs 112c, 112d, disposed on an inner surface of the first upper portion 108b and the second upper portion 110b, respectively, with the rod 106 such that the rod 106 is fixed within the receiver 102 and extends through the second opening 102b and the third opening 102c.

At step 820, the user may attach a cap 700 onto the first upper portion 108b and the second upper portion 110b to enclose the fourth opening 102d present on the proximal face of the receiver 102. In some embodiments of the present disclosure, the cap 700 may be snap fitted or screwed onto or secured in slots, grooves or holes 202a, 202b in on an outer faces of each of the first upper portion 108b and the second upper portion 110b.

FIG. 9A illustrates a side view of the receiver 900, without a rod in place, according to an embodiment of the present disclosure. As illustrated in FIG. 9A, a first outside portion 902 is connected to a second inside portion 904 using a pivot pin 910 that extends across the receiver from a first hinge 906 to a second hinge 908. The plurality of hinged connections using a single pivot pin 910 may enable the first outside portion 902 and the second inside portion 904 to exhibit one of a neutral or aligned position or an angled position relative to each other. Specifically, in this embodiment, in the neutral or aligned position, the first outside portion 902 and the second inside portion 904 are at a substantially parallel position with respect to one another.

FIG. 9B illustrates a side view of the receiver 900 with the pedicle screw 920, and without a rod in place, according to an embodiment of the present disclosure. As illustrated in FIG. 9B, the head 922 of the pedicle screw 920 includes flats on both sides which provides for only uni-axial movement, and a slit 924 that aligns with the pivot pin 910. Specifically, the slit 924 in the head of the pedicle screw 920, when inserted into the receiver 900, is configured to align with the pivot pin 910 such that the pivot pin 910 sits within the slit 924 and limits movement of the pedicle screw 920 relative to the receiver 900. Removal of flats on the sides of the head 922 could allow for true polyaxial movement of the pedicle screw 920 within the receiver 900.

To ensure only uni-axial movement of the pedicle screw 920, the receiver may include reciprocal flat portions on the lower inside wall of the first outside portion 902 and the lower inside wall of the second inside portion 904. Including a flat portion on only one of the lower inside wall of the first outside portion 902 or the lower inside wall of the second inside portion 904 would provide for a favored angle for the pedicle screw 920. Removal of both flats such that the lower inside wall of the first outside portion 902 and the lower inside wall of the second inside portion 904 are each rounded to conform to a rounded head 922 of the pedicle screw 920 would provide for polyaxial motion of the pedicle screw 920 within the receiver 900.

The pivot pin 910 may also include an internal spring to provide for an automatic return to the neutral position upon release of the first outside portion 902 and the second inside portion 904 after insertion of the head 922 of the pedicle screw 920 into the receiver 900.

FIG. 9C illustrates a front view of the receiver 900 with the pedicle screw 920 of FIGS. 9A and 9B, and without a rod in place, according to an embodiment of the present disclosure.

FIG. 9D illustrates a perspective view of the receiver 900 and pedicle screw 920 of FIGS. 9A and 9B.

FIGS. 10A-10C illustrate the method of assembling the pedicle screw 920 and the receiver 900. In FIG. 10A, pressure is placed on the top portion of the first outside portion 902 and the top portion of the second inside portion 904, causing the top of the first outside portion 902 and the top of the second inside portion 904 to pivot around pin 908 and 906 (not shown) and come together at point 926. This compression of the tops of the receiver, expands the bottom of the receiver, allowing the head 922 of the pedicle screw 920 to be inserted through the bottom of the receiver 900 as illustrated in FIG. 10B. Upon completion of the insertion of the head 922 of the pedicle screw 920 into the bottom of the receiver 928, as illustrated in FIG. 10B, the pressure is released from the top portion of the first outside portion 902 and the top portion of the second inside portion 904, resulting in the top of the first outside portion 902 and the top of the second inside portion 904 to automatically return to the neutral upright position via the spring in the pivot pin 910 (not shown), as depicted at point 926, thereby locking the head 922 of the pedicle screw 920 in the receiver 900 above and resting against the bottom of the receiver 928.

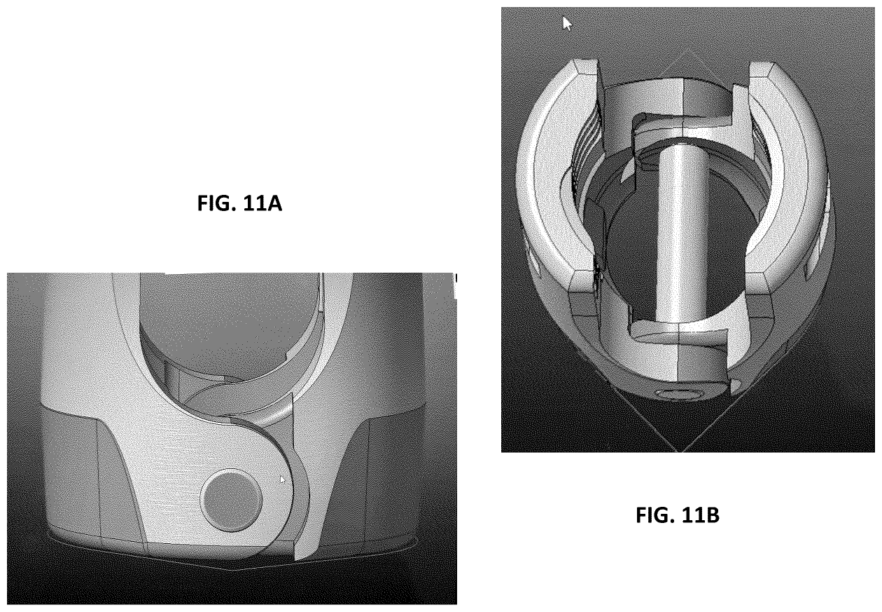

Additional illustrations of the receiver 900 and the pivot pin 910 are depicted in FIGS. 11A and 11B.

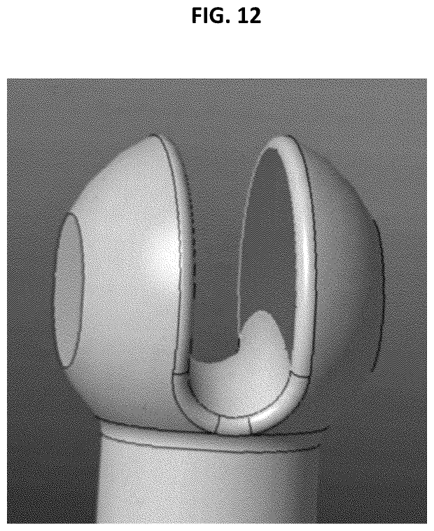

Additional illustrations of the head 922 of the pedicle screw 920 and illustrating the flats on the head 922 are depicted in FIG. 12.

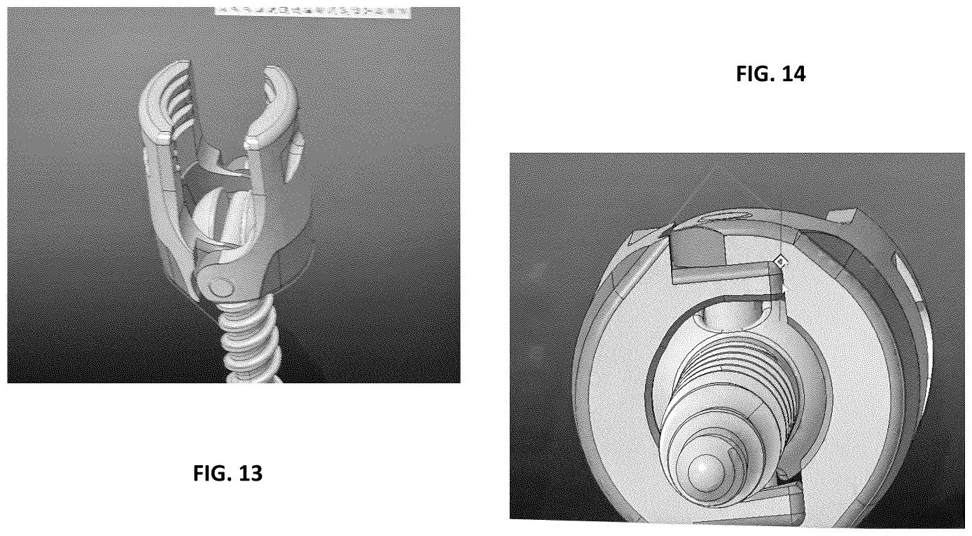

FIG. 13 illustrates a perspective view of the interior of the receiver 900 and pedicle screw 920 in an assembled state, and depicting the pivot pin 910 positioned in the slit 924 in the head 922 of the pedicle screw 920.

FIG. 14 illustrates the bottom of the receiver 900 in a closed position after completion of the assembly of the receiver 900 and the pedicle screw 920. This shows the opening in the bottom of the receiver being of a smaller diameter than the diameter of the head 922 of the pedicle screw 920, thereby preventing disassembly of the receiver 900 and the pedicle screw 920 upon insertion of a rod in the top of the receiver 900, as the rod will prevent the upper wall of the first outside portion 902 and the upper wall of the second inside portion 904 from being compressed inwardly.

Although certain embodiments have been depicted and described in detail herein, it will be apparent to those skilled in the relevant art that various modifications, additions, substitutions and the like can be made without departing from the spirit of the disclosure, and these are, therefore, considered to be within the scope of the disclosure, as defined in the following claims. Features or functionality described with respect to certain example embodiments may be combined and sub-combined in and/or with various other example embodiments. Also, different embodiments and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, can be at least partially performed via at least one entity or actor in any manner.

The terminology used herein can imply direct or indirect, full or partial, temporary or permanent, action or inaction. For example, when an element is referred to as being “on,” “connected” or “coupled” to another element, then the element can be directly on, connected or coupled to the other element and/or intervening elements can be present, including indirect and/or direct variants. In contrast, when an element is referred to as being “directly connected” or “directly coupled” to another element, there are no intervening elements present.

Although the terms first, second, etc. can be used herein to describe various elements, components, regions, layers and/or sections, these elements, components, regions, layers and/or sections should not necessarily be limited by such terms. These terms are used to distinguish one element, component, region, layer or section from another element, component, region, layer or section. Thus, a first element, component, region, layer, or section discussed below could be termed a second element, component, region, layer, or section without departing from the teachings of the present disclosure.

The terminology used herein is for describing particular example embodiments and is not intended to be necessarily limiting of the present disclosure. As used herein, the singular forms “a,” “an” and “the” are intended to include the plural forms as well, unless the context clearly indicates otherwise. The terms “comprises,” “includes” and/or “comprising,” “including” when used in this specification, specify the presence of stated features, integers, steps, operations, elements, and/or components, but do not preclude the presence and/or addition of one or more other features, integers, steps, operations, elements, components, and/or groups thereof.

As used herein, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Unless otherwise defined, all terms (including technical and scientific terms) used herein have the same meaning as commonly understood by one of ordinary skill in the art to which this disclosure belongs. The terms, such as those defined in commonly used dictionaries, should be interpreted as having a meaning that is consistent with their meaning in the context of the relevant art and should not be interpreted in an idealized and/or overly formal sense unless expressly so defined herein.

As used herein, the term “about” and/or “substantially” refers to a +/−10% variation from the nominal value/term. Such variation is always included in any given.

If any disclosures are incorporated herein by reference and such disclosures conflict in part and/or in whole with the present disclosure, then to the extent of conflict, and/or broader disclosure, and/or broader definition of terms, the present disclosure controls. If such disclosures conflict in part and/or in whole with one another, then to the extent of conflict, the later-dated disclosure controls.

The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

Whereas many alterations and modifications of the present invention will no doubt become apparent to a person of ordinary skill in the art after having read the foregoing description, it is to be understood that the particular embodiments shown and described by way of illustration are in no way intended to be considered limiting.

1. A receiver for a spinal alignment system, the receiver comprising:
a first outside portion comprising a first pair of legs, a first lower portion and a first upper portion, wherein the first pair of legs comprises a first leg and a second leg;
a second inside portion comprising a second pair of legs, a second lower portion and a second upper portion, wherein the second pair of legs comprises a third leg and a fourth leg;
a first tab positioned between the first leg and the second leg and coupled to the first lower portion;
a second tab positioned between the third leg and the fourth leg and coupled to the second lower portion;
a first aperture positioned between the first tab and the second tab;
a second aperture positioned on a first side of the receiver between the first upper portion and the second upper portion;
a third aperture positioned on a second side of the receiver between the first upper portion and the second upper portion;
a fourth aperture positioned at the top of the receiver between a first top of the first upper portion and a second top of the second upper portion;
a first hinge; and
a second hinge, wherein the first outside portion is coupled to the second inside portion via the first hinge connecting the first leg to the third leg and the second hinge connecting the second leg to the fourth leg, wherein the first hinge and the second hinge provide a range of movement for the first outside portion and the second inside portion relative to each other from a first closed position to a second open position.

2. The receiver of claim 1, wherein the first closed position results in the retention of a head of a pedicle screw in the first aperture.

3. The receiver of claim 1, wherein the second open position is achieved by moving the first upper portion toward the second upper portion.

4. The receiver of claim 1, wherein the second open position is achieved by moving the first upper portion away from the second upper portion.

5. The receiver of claim 1, further comprising a third tab disposed on or coupled to a first inner surface of the first upper portion and a fourth tab disposed on or coupled to a second inner surface of the second upper portion.

6. The receiver of claim 1, further comprising a retaining means that is adapted to couple the first upper portion to the second upper portion to retain the first upper portion and the second upper portion in the first closed position.

7. The receiver of claim 1, wherein, in the contracted position, the first upper portion and the second upper portion lean inwards towards each other or lean outwards away from each other, and (ii) in the neutral position, the first upper portion and the second upper portion are at a substantially parallel position with respect to one another.

8. The receiver of claim 1, wherein the receiver has a central portion, a distal face, and a proximal face such that the first aperture is defined at the distal face, the second aperture and the third aperture are defined at a central portion and the fourth aperture is defined at the proximal face.

9. The receiver of claim 5, wherein the fourth aperture is adapted to receive a rod such that the rod is engaged by way of the third tab and the fourth tab when the first upper portion and the second upper portion are in the neutral position.

10. The receiver of claim 1, wherein the first aperture is adapted to receive a head portion of a pedicle screw when the first upper portion and the second upper portion are in the contracted position such that upon movement of the first upper portion and second upper portion to the neutral position, the first tab and the second tab retains the head portion within the second opening.

11. The receiver of claim 1, wherein the fourth aperture is adapted to receive a cap having an open center portion such that the cap is coupled to the proximal face of the receiver onto the first upper portion and the second upper portion.

12. The receiver of claim 1, wherein the first tab and the second tab in the neutral position form a plurality of upward facing lips at a bottom of the receiver.

13. The receiver of claim 12, wherein the plurality of upward facing lips are adapted to be engaged with the head portion of the pedicle screw when the head portion is a mushroom shaped head.

14. The receiver of claim 1, further comprising a locking washer, wherein a threaded portion of a pedicle screw is inserted through the locking washer such that the locking washer rests proximate to a head portion of the pedicle screw when the receiver is coupled to the pedicle screw.

15. The receiver of claim 1, further comprising a c-ring, wherein a threaded portion of a pedicle screw is inserted through the c-ring such that the c-ring rests proximate to a head portion of the pedicle screw when the receiver is coupled to the pedicle screw.

16. The receiver of claim 1, wherein each leg of the first and second pair of legs has a horizontal portion and a vertical portion such that (i) the horizontal portion of each leg of the first pair of legs and the second pair of legs comprising at least one through hole (ii) the horizontal portion of each leg of the first pair of legs comprising a slot on an outer surface, (iii) the horizontal portion of each leg of the second pair of legs comprising a slot on an inner surface.

17. The receiver of claim 1, wherein the plurality of hinged connections comprising a plurality of wire springs and a plurality of pivot pins, respectively, wherein, to couple the first pair of legs with the second pair of legs, the at least one through hole of the first pair of legs is aligned with the at least one through hole of the second pair of legs such that the at least one through hole receives a wire spring of the plurality of wire springs and a pivot pin of the plurality of pivot pins.

18. The receiver of claim 17, wherein each wire spring of the plurality of wire springs comprising a plurality of upright portions and a central portion such that the plurality of upright portions are engaged within the slots provided on the outer surface of the first and second pair of legs when the first and second pair of legs are aligned.

19. The receiver of claim 1, wherein the first leg and the second leg of the first pair of legs are integrally connected to form a U-shaped structure and the third leg and the fourth leg of the second pair of legs are integrally connected to form a U-shaped structure.

20. The receiver of claim 1, wherein the vertical portion of the first leg and the second leg of the first pair of legs and the third leg and the fourth leg of the second pair of legs comprising a horizontal slot at about the upper end on the outer surface.

21. The receiver of claim 1, wherein the vertical portions of the first leg and the second leg of the first pair of legs and the third leg and the fourth leg of the second pair of legs comprising a horizontal slot at about the upper end on the outer surface to accept one or more tools therein.

22. A spinal alignment system comprising:
a receiver comprising a first pair of legs and a second pair of legs coupled to each other by way of a plurality of hinged connections that enables the first pair of legs and the second pair of legs to exhibit one of, a neutral position or a contracted position, wherein the first pair of legs and the second pair of legs in the neutral position defines a first opening, a second opening, and a third opening;
one or more tabs disposed onto an inner surface of each leg of the first pair of legs and the second pair of legs;
a retainer that is adapted to couple at least one leg of the first pair of legs with at least one leg of the second pair of legs to retain the first and second pair of legs in the neutral position; and
a pedicle screw having a threaded portion for securing the pedicle screw to a vertebra and a head portion coupled to the threaded portion, wherein the head portion of the pedicle screw is inserted into the first opening when the first pair of legs and the second pair of legs are in the contracted position such that upon release of the first pair of legs and the second pair of legs, the first pair of legs and the second pair of legs return to the neutral position and hold the head portion within the first opening;
a rod adapted to be inserted inside the second opening, wherein the rod is engaged by way of the one or more tabs when the first pair of legs and the second pair of legs are in the neutral position.

23. The spinal alignment system of claim 22, wherein the receiver has a distal face, a central portion, and a proximal face such that the first opening, the second opening, and the third opening are defined at the distal face, the central portion, and the proximal face, respectively.

24. The spinal alignment system of claim 22, wherein, (i) in the contracted position, the first pair of legs and the second pair of legs are bent inwards towards each other and (ii) in the neutral position, the first pair of legs and the second pair of legs are at a substantially parallel position with respect to one another.

25. A method for assembling a spinal alignment system, the method comprising:
contracting a proximal face of a receiver by applying an external force on a first pair of legs and a second pair of legs of the receiver such that the first pair of legs and the second pair of legs are bent towards each other to exhibit a contracted position thus expanding a first opening present on a distal face of the receiver, wherein the contraction is enabled by way of a plurality of hinged connections attaching the first pair of legs and the second pair of legs;
inserting a head portion of a pedicle screw inside the first opening;
releasing the external force applied on the first pair of legs and the second pair of legs such that the first pair of legs and the second pair of legs return to a neutral position such that the head portion of the pedicle screw is held therein;
inserting a rod inside a second opening present in a central portion of the receiver;
engaging one or more tabs disposed on an inner surface of each leg of the first pair of legs and the second pair of legs with the rod; and
attaching a cap onto a first top portion of the first pair of legs and a second top portion of the second pair of legs to enclose a third opening present on the proximal face of the receiver.
